(19) United States
(12) Patent Application Publication
(10) Pub. No.: US 2024/0158362 A1
(43) Pub. Date: May 16, 2024

(54) PROCESS FOR PREPARING ALKYL-4-OXOTETRAHYDROFURAN-2-CARBOXYLATE

(71) Applicant: Bayer Aktiengesellschaft, LEVERKUSEN (DE)

(72) Inventors: Anton LISHCHYNSKYI, Leverkusen (DE); Carlos MORIANA HERRAIZ, Leverkusen (DE); Günter HÖMBERGER, Leverkusen (DE); Mark James FORD, Leverkusen (DE)

(21) Appl. No.: 18/279,626

(22) PCT Filed: Feb. 28, 2022
(86) PCT No.: PCT/EP2022/054925

§ 371 Date: Aug. 31, 2023

(30) Foreign Application Priority Data
Mar. 3, 2021 (EP) 21160527.4

(51) Int. Cl. C07D 307/32

(52) U.S. Cl. CPC C07D 307/32

(57) ABSTRACT

The present invention relates to a novel method for preparing alkyl 4-oxotetrahydrofuran-2-carboxylate (I).

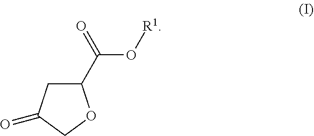

The present invention relates to a novel method for preparing alkyl 4-oxotetrahydrofuran-2-carboxylate (I).

Methyl 4-oxotetrahydrofuran-2-carboxylate of formula (I) is an important precursor of agrochemical (cf. WO2018/228985) active substances.

The synthesis of methyl 4-oxotetrahydrofuran-2-carboxylate of formula (I) is known, e.g. from Helv. Chim. Acta 1959, 1177 and WO 2016/205633. However, if starting from dimethyl (Z)-butenedioate, three reaction steps are necessary to prepare methyl 4-oxotetrahydrofuran-2-carboxylate of formula (I), which is accompanied by a loss of yield. Moreover, the reagents that are used in the prior art (for example sodium powder, NaH, TMSCHN₂, CH₂N₂) are unsuitable for an industrial-scale synthesis, since safe handling of these chemicals on a large scale is difficult or they are highly toxic.

In the light of the prior art described above, the object of the present invention is to develop, starting from compounds of general formula (II) and (III) in only two reaction steps, a method for preparing compounds of general formula (I) that is also suitable for production on a large scale.

The object described above is achieved by a method for preparing compounds of general formula (I)

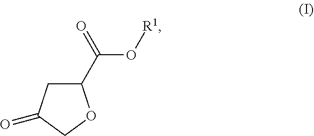

in which
R¹ is (C₁-C₄) alkyl,
characterized in that compounds of general formula (II)

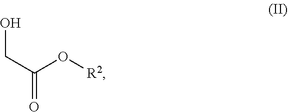

in which
R² is (C₁-C₄) alkyl,
afford with compounds of general formula (III)

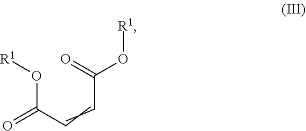

in which R¹ is as defined above,
through addition of MO-Z in Z—OH,
in which
M is an alkali metal ion and
Z is (C₁-C₄) alkyl, t-butyl excepted,
cyclization products of general formula (IV)

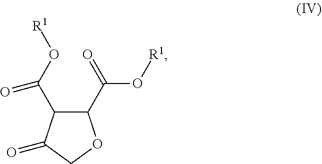

in which R¹ is as defined above,
which under non-hydrolytic conditions undergo dealkoxycarbonylation and react to form compounds of general formula (I).

Preferred definitions of the radicals for the compounds of general formulas (I), (II), (III), (IV), MO-Z and Z—OH are as follows:
R¹ is ethyl or methyl,
R² is ethyl or methyl,
M is sodium or potassium,
Z is ethyl or methyl.

Particularly preferred definitions of the radicals for the compounds of general formulas (I), (II), (III), (IV), MO-Z and Z—OH are as follows:
R¹ is methyl,
R² is methyl,
M is sodium,
Z is methyl.

Elucidation of the Method

The reaction conditions for preparing compounds of general formula (I) are elucidated in detail hereinbelow.

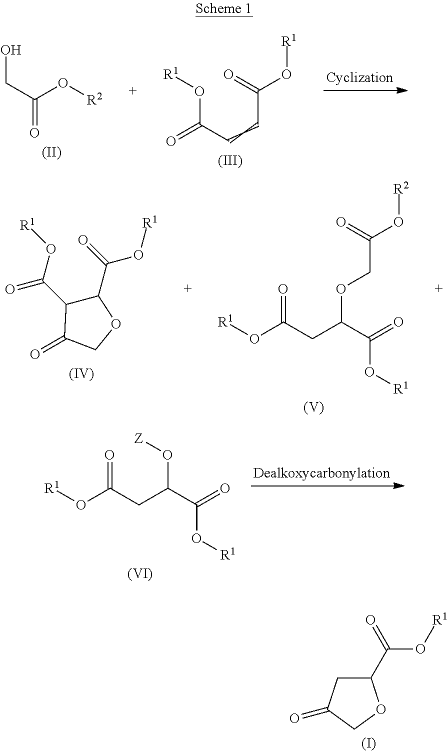

The compounds of general formula (II) react with compounds of general formula (III) in the presence of MO-Z in Z—OH to form cyclization products of general formula (IV), which under non-hydrolytic conditions undergo dealkoxycarbonylation and react to form compounds of general formula (I).

During the cyclization, in addition to the actual product there may also be present in the reaction mixture compounds of general formula (IV) and also residual intermediates of general formulas (V) and (VI), which under the given reaction conditions react further to form the product of general formula (IV).

The compounds of general formula (II) and (III) are commercially available. The compounds of general formula (III) can surprisingly be used in the form of the E isomer or the Z isomer and lead to a comparable result. This is not known from the literature. Under the reaction conditions, an isomerization between the E isomer and the Z isomer takes place.

The compounds of general formula (I) have a stereocentre. The product is consequently present in the form of a racemate.

Cyclization:

The cyclization is known from the prior art, where it is carried out using NaH or sodium powder (Helv. Chim. Acta 1959, 1177; WO 2016/205633). These reagents are unsuitable for an industrial-scale synthesis, since their safe handling on a large scale is difficult.

The yield of the reaction according to the invention is higher (>30%) than that of the reaction using NaH or sodium powder described in the prior art (<30%). Moreover, the use of the alkoxide base means that the reaction can be employed on an industrial scale too.

Advantageous for achieving a high yield is the slow addition of the alkoxide base, for example through metered addition.

In addition, the equilibrium of the reaction to form the product can be shifted when the alcohol used as solvent and additionally formed during the cyclization is removed by distillation. The yield increases.

The molar ratio of MO-Z to compounds of general formula (II) is 0.8 to 3 equivalents, preferably 0.9 to 1.2 equivalents.

The molar ratio of Z—OH to compounds of general formula (II) is 1 to 10 equivalents, preferably 4 to 7 equivalents.

The molar ratio of compounds of general formula (II) to compounds of general formula (III) is 0.8 to 3 equivalents, preferably 0.9 to 1.2 equivalents.

The temperature may be varied within a wide range and depends for example on the solvent. It is for the reaction preferably 15 to 70° C., particularly preferably 40 to 60° C.

The reaction is normally carried out in a solvent, preferably in anisole, THF, toluene, xylene or Me-THF.

The solvent may contain Z—OH. Preferably, this is an anhydrous (“dry” or absolute) solvent.

Dealkoxycarbonylation (Organic Reactions, Vol. 81):

Whereas with sulfuric acid in water (see Helv. Chim. Acta 1959, 1177; WO 2016/205633), ester cleavage/decarboxylation to 4-oxotetrahydrofuran-2-carboxylic acid takes place, under non-hydrolytic, e.g. anhydrous, reaction conditions, the ester in compounds of general formula (I) remains present, which means there is no need for the additional step of renewed esterification, which in the prior art is carried out with CH₂N₂. There is consequently also no need for the numerous extractions necessary in the prior art of the 4-oxotetrahydrofuran-2-carboxylic acid, which is difficult to isolate from water.

The yield can consequently be increased substantially (>95% versus 75% with sulfuric acid in the prior art). Toxic reagents such as diazomethane can be dispensed with.

The reagent (see Table 1) is used in excess optionally in combination with a solvent. Preferably, the reagent is also used as the solvent.

The temperature for the reaction depends on the reagent/solvent.

Table 1 lists some of these reaction conditions by way of example, but without any limitation thereto.

TABLE 1
Dealkoxycarbonylation

| Conditions | | |
|---|---|---|
| (Reagent = solvent) | Temp./° C. | Time/hours |
| Acetic acid | 118 | 6 |
| Acetic acid/trifluoroacetic acid | 115-118 | 25 |
| Propionic acid | 140 | 18 |
| B(OH)₃ | 145-175 | 4 |

### EXAMPLES

The present invention is elucidated in more detail by the examples that follow, without restriction of the invention thereto.

Measurement Methods

The products were characterized by ¹H NMR.

### Example 1

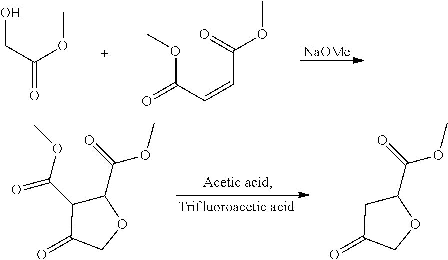

### Methyl 4-oxotetrahydrofuran-2,3-dicarboxylate

356 g of methyl glycolate (3.95 mol) and 541 g of dimethyl maleate (3.75 mol) are added together with 2.175 l of anisole to a vessel equipped with a heating/cooling jacket (6 l). The mixture is heated to 49° C. and then 783 g of 30% sodium methoxide (NaOMe=NaOMethyl) in methanol is added over 6 hours. During the first minutes, the internal temperature rises to 55° C. and then remains at 50° C. After approx. 30% of the base has been added, the reaction mixture becomes turbid and a solid precipitates. At the end of the addition of the base, a brownish suspension that is still easily stirrable is present. The reaction mixture is cooled to 20° C. and stirred overnight. The next day, the reaction mixture is heated to 50° C. (55° C. heating jacket) and 849 g of methanol is over a period of 6 hours removed by distillation at a pressure of 360 mbar (at the beginning) to 60 mbar (at the end). The reaction mixture is then cooled to 20° C. and stirred overnight. This reaction mixture is then added to a mixture of dilute sulfuric acid prepared from 2.373 l of water and 225 g of concentrated sulfuric acid (2.17 mol) contained in a vessel equipped with a heating/cooling jacket (6 l), at 10° C. The reaction mixture is stirred for 2 hours at 10 to 12° C., then warmed to 20° C. The two-phase mixture is then separated and the aqueous phase is extracted with anisole. The combined organic phases are concentrated to 990 g.

### Methyl 4-oxotetrahydrofuran-2-carboxylate

The oil from the previous reaction step is added to 1200 ml of acetic acid. To this is added 67.6 g of trifluoroacetic acid and the reaction mixture is stirred at 115 to 118° C. for 24 hours. The acetic acid is then distilled off and the product is purified by distillation. The product is an oil (350 g, 66% yield).

NMR Data

¹H-NMR (600 MHz, DMSO-d6): δ (ppm)=4.97 (dd, J=8.8, 5.1 Hz, 1H), 4.02 (d, J=16.7 Hz, 1H), 3.99 (d, J=16.7 Hz, 1H), 3.69 (s, 3H), 2.88 (dd, J=18.2, 8.8 Hz, 1H), 2.60 (dd, J=18.2, 5.1 Hz, 1H).

TABLE 2
Comparison of the yield

| | Reaction conditions | Overall yield |
|---|---|---|
| Example No. 1 | NaOMe/acetic | 66% |
| | acid/trifluoroacetic acid | |
| Comparative | Sodium powder, | 23% |
| example: Helv. | sulfuric acid, water, | |
| Chim. Acta 1959, | CH₂N₂ | |
| 1177 | | |

1. A method for preparing compounds of general formula (I) 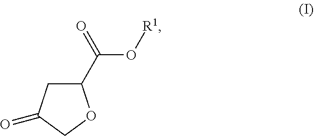
in which
R¹ is (C₁-C₄) alkyl,
characterized in that compounds of general formula (II) 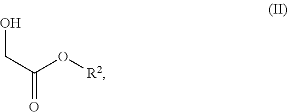
in which
R² is (C₁-C₄) alkyl,
are reacted with compounds of general formula (III) 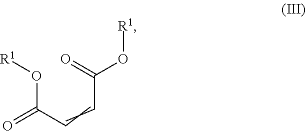
in which R¹ is as defined above,
in the presence of MO-Z in an alcohol Z—OH,
in which
M is an alkali metal ion and
Z is (C₁-C₄) alkyl, t-butyl excepted,
cyclization products of general formula (IV) are formed in a cyclization reaction 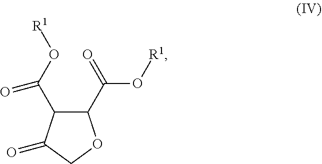
in which R¹ is as defined above,
which under non-hydrolytic conditions the cyclization products undergo dealkoxycarbonylation and react to form compounds of general formula (I).

2. The method according to claim 1, characterized in that:
R¹ is ethyl or methyl,
R² is ethyl or methyl,
M is sodium or potassium,
Z is ethyl or methyl.

3. The method according to claim 2, characterized in that:
R¹ is methyl,
R² is methyl,
M is sodium,
Z is methyl.

4. The method according to claim 1, characterized in that, in addition to the cyclization product having the general formula (IV), the intermediates (V) and (VI) also form, which can likewise react further to form compounds of formula (IV).

5. The method according to claim 1, characterized in that the alcohol Z—OH is removed by distillation during the cyclization reaction.

6. The method according to claim 1, characterized in that the cyclization is carried out at 15 to 70° C.

7. The method according to claim 6, characterized in that the cyclization is carried out at 40 to 60° C.

8. The method according to claim 1, characterized in that a solvent for the cyclization is anisole, THF, toluene, xylene or Me-THF.

9. The method according to claim 8, characterized in that the solvent also contains Z—OH, where Z is as defined in claim 1.

10. The method according to claim 8, characterized in that the solvent is used for the cyclization is an anhydrous solvent.

11. The method according to claim 1, characterized in that the MO-Z in Z—OH is metered in.

12. The method according to claim 1, characterized in that NaOMethyl is used for the cyclization.

13. The method according to claim 1, characterized in that a reagent/solvent in the dealkoxycarbonylation is acetic acid or consists of a mixture of acetic acid and trifluoroacetic acid.
